(19) United States
(12) Patent Application Publication
(10) Pub. No.: US 2006/0136267 A1
(43) Pub. Date: Jun. 22, 2006

(54) System and method for automatic scheduling based on remote monitoring

(75) Inventors: Cameron Brackett, Overland Park, KS (US); Jana Malinowski, Platte City, MO (US); Asim Pasha, Liberty, MO (US)

Correspondence Address: SHOOK, HARDY & BACON L.L.P.;Intellectual Property Department, 2555 GRAND BOULEVARD, KANSAS CITY, MO, 64108-2613, US

(73) Assignee: Cemer innovation, Inc., Overland Park, KS (US)

(21) Appl. No.: 11/018,351

(22) Filed: Dec. 22, 2004

(51) Int. Cl. A61B 5/00; G06F 19/00

(52) U.S. Cl. 705/3; 705/8

(57) ABSTRACT

A system and method are provided for managing patient care based on data received from a remote monitoring system. The system includes a data monitor for automatically evaluating the data received from the remote monitoring system and a scheduling module for receiving a data evaluation from the data monitor. The scheduling module includes tools for adjusting a patient schedule. Schedule adjustment actions can be automatically triggered by the received data evaluation. Typically, the data monitor will compare the received data with patient medical records to evaluate the received data received from the remote monitoring system to determine if the received data meets pre-set expectations. If the received data does not meet pre-set expectations, a scheduling action will be triggered. The system will automatically notify the patient of scheduling actions.

### CROSS-REFERENCE TO RELATED APPLICATIONS

None.

### STATEMENT REGARDING FEDERALLY SPONSORED RESEARCH OR DEVELOPMENT

None.

### TECHNICAL FIELD

Embodiments of the present invention relate to scheduling of patient appointments and in particular to scheduling of patient appointments based on evaluation of remotely monitored patient information

### BACKGROUND OF THE INVENTION

In the current health care environment, systems are provided for managing scheduling of patient appointments with care providers. Often, patients with chronic or persistent conditions are scheduled for appointments at regular intervals in order to enable care providers to monitor the patients' conditions at the regularly scheduled appointments. In other words, care providers will require the patients to visit regularly so that factors such as blood pressure, heart functioning, glucose levels, or other medical characteristics may be verified at regular intervals.

Additionally, in order to facilitate monitoring of patients, remote monitoring equipment has become increasingly available. Patients having chronic conditions can be given the ability to monitor their own conditions at home. For example, diabetics can be given a home glucometer kit that can measure blood sugar level. The care provider can download remotely the remotely monitored blood sugar level from the patient remote monitoring device. Patients with pacemakers or other Implantable Cardioverter Defibrillators (ICD's) can transmit their readings to the care provider from home. Patients with such conditions are typically seen by their care providers on a regular basis.

Despite the availability of such systems, patients often report for scheduled appointments that prove to be unnecessary. For example, a patient having remote monitoring equipment such as a pacemaker may have pre-set monthly appointments that are stored in a care provider's management system. The patient visits the physician's office for the monthly appointment and a technician connects to the remote monitoring device and downloads information to determine the patient's status. If the patient's condition is normal, the technician enters the data into the care provider management system and sets up another appointment for the next visit. Had the physician known that the patient's condition was normal, the regularly scheduled monthly appointment could have been avoided.

Although provider management systems and remote monitoring systems are available today, no system is available for bridging the gap between the systems in order to allow the existing systems to communicate with one another. Today, these systems are completely separate and distinct. Thus, an individual who evaluates remote monitoring data would be required to take initiative to relate the remote monitoring data to a patient schedule and to re-schedule a patient appointment when necessary. Generally, individuals responsible for evaluating remotely monitored data are not the same individuals responsible for patient or physician scheduling.

Accordingly, a solution is needed for reducing unnecessary patient visits. Furthermore, a solution is needed for automatically evaluating remotely monitored data and for adjusting patient scheduling or treatment in accordance with the evaluation results.

### BRIEF SUMMARY OF THE INVENTION

Embodiments of the present invention are directed to a system for managing patient care based on data received from a remote monitoring system. The system includes a data monitor for evaluating the data received from the remote monitoring system and a scheduling component for receiving a data evaluation from the data monitor. The scheduling component includes tools for adjusting a patient schedule. Schedule adjustment is triggered by the received data evaluation.

In a further aspect, a system is provided for assessing patient status. The system includes a remote monitoring device connected with the patient. The remote monitoring device monitors at least one medical characteristic of the patient. The system additionally includes a data monitor connected with the remote monitoring device. The data monitor is also connected to a care provider management system. The data monitor includes components for evaluating the at least one remotely monitored medical characteristic and for conveying an evaluation to the care provider management system.

In yet a further aspect of the invention, a method is provided for managing patient care based on data received from a remote monitoring system. The method includes evaluating the data received from the remote monitoring system to determine if the received data meets pre-set expectations. The method additionally includes triggering a scheduling action in response to evaluated data that does not meet pre-set expectations.

In yet an additional aspect of the invention, a method is provided for tracking patient status using a care provider management system and a remote monitoring device connected with a patient. The remote monitoring device generates data related to at least one medical characteristic of the patient. The method includes transmitting the generated data to a data monitor in a location of a care provider. The data monitor is also connected with the care provider management system. The method additionally includes evaluating the received data from the remote monitoring system using the data monitor and conveying an evaluation from the data monitor to the care provider management system.

### BRIEF DESCRIPTION OF THE DRAWINGS

The present invention is described in detail below with reference to the attached drawings figures, wherein:

FIG. 1 is a block diagram illustrating an overview of a system in accordance with an embodiment of the invention;

FIG. 2 is a block diagram illustrating dynamic scheduling components in accordance with an embodiment of the invention;

FIG. 3 is a flow chart illustrating a procedure for evaluating remote data and keeping or removing an appointment based on the evaluation in accordance with an embodiment of the invention; and

FIG. 4 is a flow chart illustrating a technique for re-scheduling and patient notification in accordance with an embodiment of the invention.

### DETAILED DESCRIPTION OF THE INVENTION

I. System Overview

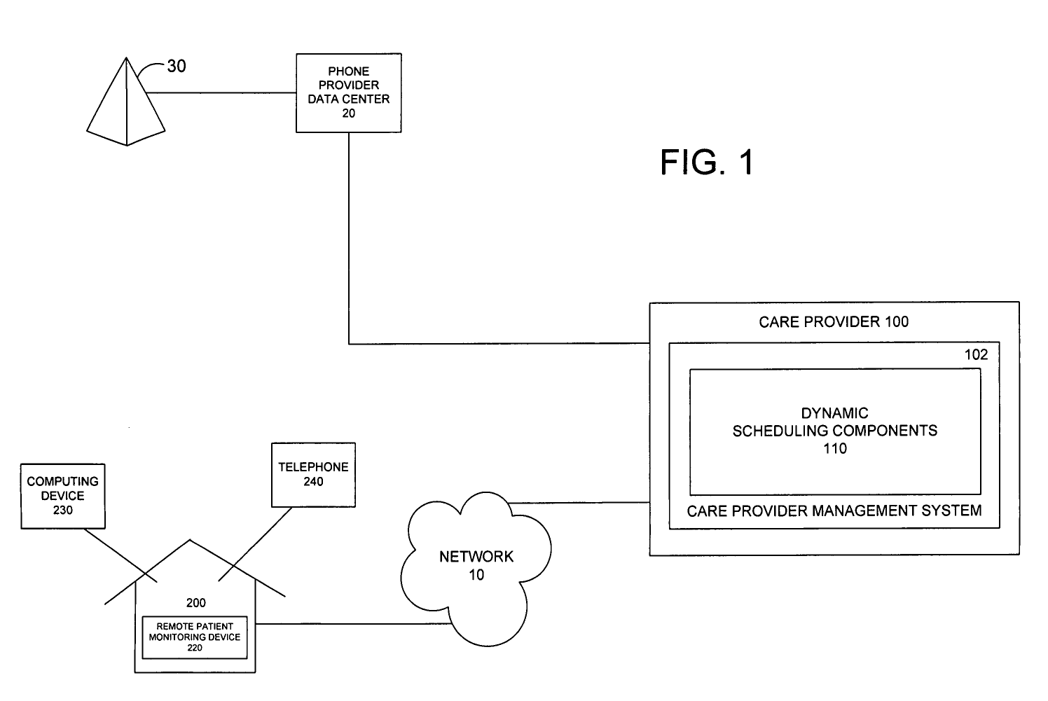

Embodiments of the invention include a method and system for automated patient scheduling based on evaluation of remote monitoring data from a remote monitoring device. FIG. 1 illustrates a system for practicing a method in accordance with an embodiment of the invention. A care provider 100 preferably operates a care provider management system 102. The care provider management system 102 preferably includes dynamic scheduling components 110. The dynamic scheduling components 110 may communicate over a network 10 with a remote patient monitoring device 220 within a patient home 200. The patient home 200 may also include a computing device 230 and a telephone 240. The care provider 100 may communicate with a phone provider data center 20 through a communications tower 30.

The dynamic scheduling components 110 are provided within the care provider management system 102 and perform scheduling functions based on an evaluation of data provided from the remote patient monitoring device 220. The care provider management system 102 operates with a computerized operating environment as will be described below.

II. Operating Environment

The automated scheduling system and method may be implemented in a suitable computing system environment associated with the care provider management system 102. The invention is described in the general context of computer-executable instructions, such as program modules, being executed by a computer. Generally, program modules include routines, programs, objects, components, data structures, etc. that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote computer storage media including memory storage devices.

An exemplary system for implementing the invention includes a general purpose-computing device including a processing unit, a system memory, and a system bus that couples various system components including the system memory to the processing unit. The computing device typically includes a variety of computer readable media. By way of example, and not limitation, computer readable media may comprise computer storage media and communication media.

The system memory includes computer storage media in the form of volatile and/or nonvolatile memory such as read only memory (ROM) and random access memory (RAM). A basic input/output system (BIOS), containing the basic routines that help to transfer information between elements within computer, such as during start-up, is typically stored in ROM. RAM typically contains data and/or program modules that are immediately accessible to and/or presently being operated on by processing unit.

The computing device may also include other removable/nonremovable, volatile/nonvolatile computer storage media. A hard disk drive may read from or write to nonremovable, nonvolatile magnetic media, a magnetic disk drive that reads from or writes to a removable, nonvolatile magnetic disk, and an optical disk drive may read from or writes to a removable, nonvolatile optical disk such as a CD ROM or other optical media. Other removable/nonremovable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive may typically be connected to the system bus through a non-removable memory interface. The magnetic disk drive and optical disk drive are typically connected to the system bus by a removable memory interface.

A user may enter commands and information into the computing device through input devices such as a keyboard and pointing device, commonly referred to as a mouse, trackball or touch pad. Other input devices may include a microphone, joystick, game pad, satellite dish, scanner, or the like. These and other input devices are often connected to the processing unit through a user input interface that is coupled to the system bus, but may be connected by other interface and bus structures, such as a parallel port, game port or a universal serial bus (USB). A monitor or other type of display device is also connected to the system bus via an interface, such as a video interface. In addition to the monitor, the computer system may also include other peripheral output devices such as speakers and printers may be connected through an output peripheral interface.

The computing devices utilized in the present invention may operate in a networked environment using logical connections to one or more remote computers that typically includes many or all of the elements described above. When used in a LAN networking environment, the computing device may be connected to the LAN through a network interface or adapter. When used in a WAN networking environment, the computing device typically includes a modem or other means for establishing communications over the WAN, such as the Internet. The modem, which may be internal or external, may be connected to the system bus via the user input interface, or other appropriate mechanism. In a networked environment, program modules may be stored in the remote memory storage device. Although the components of the computing device are not shown, those of ordinary skill in the art will appreciate that such components and the interconnection are well known.

III. System and Method of the Invention

As set forth above, FIG. 1 illustrates a system for practicing a method for automated scheduling based on evaluation of remote monitoring data in accordance with an embodiment of the invention. A care provider 100, such as a hospital or ambulatory care facility, preferably operates a care provider management system 102.

The care provider management system 102 may incorporate such systems as Cerner IQHealth and Cerner Access Management, produced by the Cerner Corporation. Features from these systems that may be incorporated will be further described below in conjunction with each of the illustrated components.

The care provider management system 102 preferably includes dynamic scheduling components 110. The dynamic scheduling components 110 are capable of communicating over the network 10 with the remotely located patient monitoring device 220 within the patient home 200. The remotely located monitoring device 220 may be a device such as a home glucometer kit or an ICD that can provide updates on a patient's condition. The network 10 may be any type of network such as those described above in conjunction with the description of the operating environment.

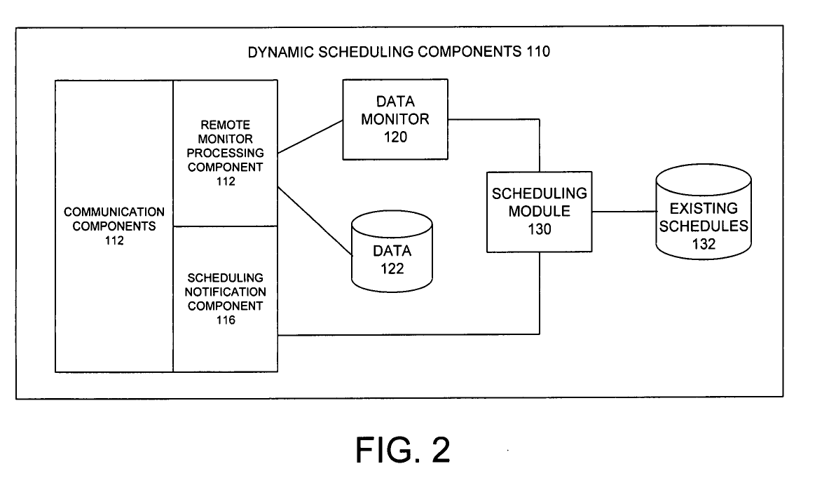

As illustrated in FIG. 2, the dynamic scheduling components 110 may include communication components 112, a remote monitor processing component 114, and a scheduling notification component 116. The dynamic scheduling communication components 110 may further include a data monitor 120 and a data storage area 122. The dynamic scheduling components 110 may further include a scheduling component 130 that is able to access existing schedules 132.

Communication components 112 allow the dynamic scheduling components 110 to receive monitoring information from the remote patient monitoring device 220 and to communicate scheduling information to the patient through the use of an email message to the remote computing device 230 or a telephone call via the home provider data center 20 and telephone network 30 to the home telephone 240.

The remote monitor processing component 114 processes and interprets data received through the communication components 112 from the remote patient monitoring device 220. The remote monitor processing component 114 passes the interpreted data to the data monitor 120.

The data monitor 120 evaluates the data received from the remote monitor processing component 114. The data monitor 120 is capable of accessing stored data 122. The stored data 122 may include a Personal Health Record (PHR) that includes data related to each individual patient or participant. The stored data 122 may include information on allergies, immunizations, measurements, conditions, tests, medications, surgeries, or procedures. The stored data 122 may also include an index such as an Enterprise Management Person Index (EMPT) provided with the Cerner Access Management System. The EMPI facilitates safe care by supplying complete and accurate information regarding a patient's current and past medical conditions and patient norms.

Accordingly the data monitor 120 may evaluate the data received from the remote monitor processing component 114 to determine if the patient's data is normal compared with the stored data 122. Evaluation of data may include checking of parameters such as blood pressure or glucose levels, but may include further steps. For instance, the data monitor 120 may check patient records to determine the types of medication that a patient takes and the side effects of the medication in order to give full consideration to personalized norms. The data monitor 120 may evaluate the data to determine if a patient's medication dosage is excessive or inadequate. If the patient's data is normal, the data monitor 120 may determine that a currently scheduled appointment is unnecessary and forward this information to the scheduling module 130.

The scheduling module 130 accesses a storage area containing existing patient schedules 132. The scheduling module 130 is capable of canceling an existing appointment contained within the existing schedules storage area 132. The scheduling module 130 may also be capable of scheduling a new appointment and storing the appointment in the patient's existing schedule 132. The scheduling module may include components such as an Enterprise Scheduling Management (ESM) component provided by Cerner's Access Management System.

The scheduling module 130 may also communicate with other components of the care provider management system if necessary. For example, it may be necessary to bump patients or revise the schedules of other patients in order to accommodate a single remotely monitored patient. In some instances, it may be necessary to page a doctor to accommodate an emergency. The scheduling module 130 may automatically schedule a room for a patient in case of emergency. Furthermore, in embodiments of the invention, the scheduling module may deliver a remotely monitored patient's medical chart to an appropriate doctor. If a doctor is on vacation, the scheduling module 130 may select and contact an alternate physician.

The scheduling module 130 may make patient visits more efficient for provider by transmitting all necessary data as part of scheduling a visit. Message templates such as those provided by Cerner's IQ Health solution can be used to save time and promote privacy. Accordingly, the patient may avoid manually filling out forms. The scheduling module 130 can also revise a patient's schedule of medications or revise orders based on evaluation of the remote monitoring data provided by the data monitor 120. The scheduling module 130 reports its re-scheduling actions to the scheduling notification component 116.

The scheduling notification component 116 communicates scheduling changes to the remotely located patient. The scheduling notification component 116 may utilize the communication components 112 to send an email message over the network 110 to the patient's home computing device 230. The home computing device 230 may be a personal computer, a personal digital assistant (PDA) or other device connected with the caregiver management system 102. The scheduling notification component 116 may further utilize the communication components 112 to communicate scheduling changes to the patient's home phone 240 using the phone provider data center 20 and communication tower 30. The scheduling notification preferably implements secure messaging components such as those provided by Cerner's IQ Health system in order to increase efficiency and guarantee privacy.

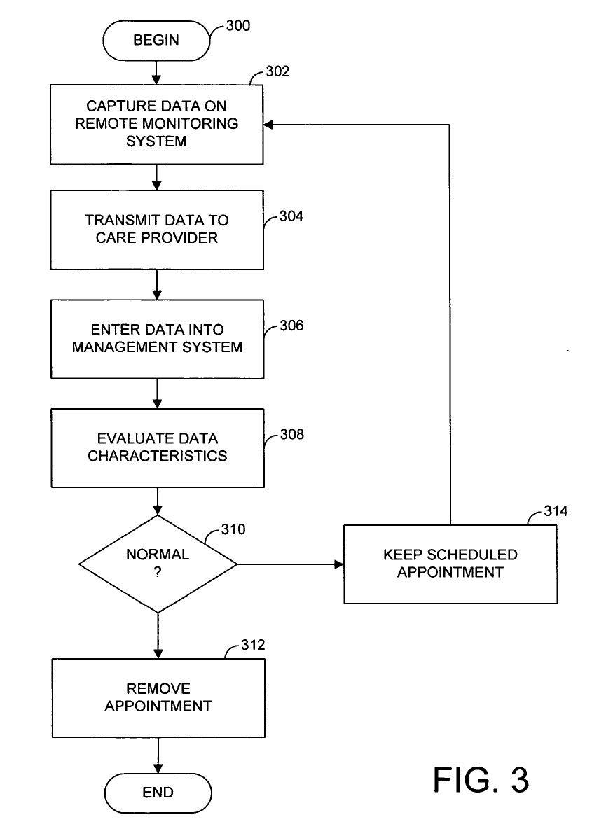

FIG. 3 is a flow chart illustrating a method for determining if a patient appointment should be kept based on data received from remote monitoring equipment.

The process begins in step 300 and the remote patient monitoring device 220 captures data in step 302. In step 304, the remote patient monitoring device 220 transmits captured data to the care provider. Using the components described above, the received data is entered into the care provider management system in step 306. In step 308, the data monitor 120 evaluates the characteristics of the received data. As set forth above, the data monitor 120 may compare the received data to patient norms.

If the data monitor 120 determines that the data received is normal in step 310, the data monitor 120 sends this information to the scheduling module 130 so that the scheduling module 130 can remove the appointment in step 312. If the data monitor 120 determines that the received data is not within a normal range in step 310, the scheduled appointment remains scheduled in step 314. In some embodiments of the invention, the scheduling module 130 may receive confirmation that the scheduled appointment will be kept and may send a notification to the patient that the patient should report for the scheduled appointment. In other embodiments, the system may assume that the patient will report for the scheduled appointment and will not send any further notification to the patient. After step 314, the system may return to step 302 to continue the ongoing monitoring process.

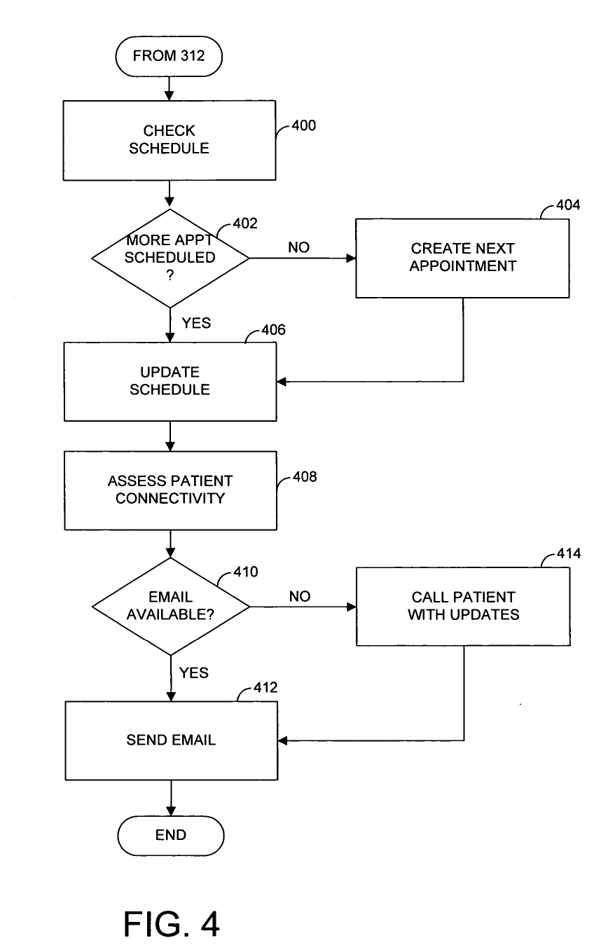

FIG. 4 is a flow chart illustrating a process for scheduling after removal of the appointment in step 312. In step 400, the scheduling module 130 checks the patient schedule. If no more appointments are scheduled, the scheduling module 130 creates a next appointment in step 404. In step 406, the scheduling module 130 updates the schedule. If in step 402, more appointments have been scheduled, the scheduling module 130 continues directly to step 406 to update the patient schedule with the appointment removal. In step 408, the scheduling notification component 116 assesses patient connectivity. If the patient has email available in step 410, the scheduling notification component 116 sends email to the patient with the updated schedule in step 412. If the patient does not have email available, the scheduling notification component 116 notifies the patient by phone in step 414.

As an alternative to the notification procedures provided above, the dynamic scheduling components 110 may post a scheduling change notification to a patient calendar contained within a provider management system 102 and available to the patient. The dynamic scheduling components 110 may also download the scheduling notification to a patient PDA that functions within the care provider management system 102.

For chronically ill patients who are on a regular doctor visit schedule and have remote monitoring capabilities, the solution offers the ability to auto-reschedule an appointment based on data being remotely monitored from the patient. The system and method in accordance with embodiments of the invention will eliminate wasted care provider and patient time by eliminating excessive appointments using remote monitoring capabilities of devices interfaced with a care provider management system to enter the data directly. As set forth above, the captured data is evaluated against a set of parameters to determine if the values are within acceptable limits. If they are, then the patient's appointment is automatically cancelled and rescheduled for the following month or other appropriate time frame. Once rescheduled, a notification is sent back to the patient using secure components or is posted on a patient calendar or downloaded to a patient PDA. Dynamic scheduling based on remote monitoring data allows physicians to optimize their schedules by reducing unnecessary visits. Although the system and method above are described in conjunction with remote monitoring from a patient's home, the monitoring could also occur in additional settings such as a school or an office.

While particular embodiments of the invention have been illustrated and described in detail herein, it should be understood that various changes and modifications might be made to the invention without departing from the scope and intent of the invention. The embodiments described herein are intended in all respects to be illustrative rather than restrictive. Alternate embodiments will become apparent to those skilled in the art to which the present invention pertains without departing from its scope.

From the foregoing it will be seen that this invention is one well adapted to attain all the ends and objects set forth above, together with other advantages, which are obvious and inherent to the system and method. It will be understood that certain features and sub-combinations are of utility and may be employed without reference to other features and sub-combinations. This is contemplated and within the scope of the appended claims.

What is claimed is:

1. A system for managing patient care based on data received from a remote monitoring system, the system comprising:
a data monitor for evaluating the data received from the remote monitoring system; and
a scheduling module for receiving a data evaluation from the data monitor, the scheduling component including tools for adjusting a patient schedule, wherein adjustment is triggered by the received data evaluation.

2. The system of claim 1, further comprising a data storage area, the data storage area including patient medical records.

3. The system of claim 2, wherein the data storage area further comprises a personal health record for each participating patient.

4. The system of claim 3, wherein the data monitor further comprises tools for comparing the data received from the remote monitoring system with information in the data storage area.

5. The system of claim 1, further comprising a scheduling storage area for storing patient scheduling information.

6. The system of claim 5, wherein the scheduling storage area further comprises physician scheduling information.

7. The system of claim 1, wherein the scheduling module comprises tools for adjusting a patient appointment schedule.

8. The system of claim 1, wherein the scheduling module comprises tools for revising a patient medication schedule.

9. The system of claim 1, further comprising a scheduling notification component for providing notification to a patient upon schedule adjustment.

10. The system of claim 9, wherein the scheduling notification component notifies the patient using one of email and telephone communications.

11. A method for managing patient care based on data received from a remote monitoring system, the method comprising:
evaluating the data received from the remote monitoring system to determine if the received data meets pre-set expectations; and
triggering a scheduling action in response to evaluated data that does not meet pre-set expectations.

12. The method of claim 11, wherein evaluating the data comprises accessing information in a data storage area, the information including patient medical records.

13. The method of claim 12, wherein evaluating the data comprises accessing information including a personal health record from the data storage area for a monitored patient.

14. The method of claim 13, further comprising comparing the data received from the remote monitoring system with information in the data storage area in order to determine whether the evaluated data meets the pre-set expectations.

15. The method of claim 14, further comprising accessing scheduling storage area for storing patient scheduling information.

16. The method of claim 15, further comprising adjusting a patient appointment schedule using a scheduling module.

17. The method of claim 16, wherein adjusting a patient appointment schedule comprises canceling a patient appointment.

18. The method of claim 15, further comprising adjusting a patient medication schedule using a scheduling module.

19. The method of claim 11, further comprising providing scheduling notification to a patient upon triggering a scheduling action.

20. The method of claim 19, wherein providing scheduling notification comprises notifying the patient using one of email and telephone communications.

21. A system for assessing patient status, the system comprising:
a remote monitoring device connected with the patient, the remote monitoring device monitoring at least one medical characteristic of the patient; and
a data monitor connected with the remote monitoring device and to a care provider management system, the data monitor including components for evaluating the at least one remotely monitored medical characteristic and for conveying an evaluation to the care provider management system.

22. The system of claim 21, further comprising a data storage area including patient medical records.

23. The system of claim 22, wherein the data monitor further comprises tools for comparing the data received from the remote monitoring system with the patient medical records in the data storage area.

24. The system of claim 21, further comprising a scheduling storage area for storing patient scheduling information.

25. The system of claim 24, wherein the scheduling module comprises tools for adjusting a patient appointment schedule.

26. The system of claim 24, wherein the scheduling module comprises tools for revising a patient medication schedule.

27. The system of claim 25, further comprising a scheduling notification component for providing notification to a patient upon schedule adjustment.

28. A method for tracking patient status using a care provider management system and a remote monitoring device connected with a patient that generates data related to at least one medical characteristic of the patient, the method comprising:
transmitting the generated data to a data monitor in a location of a care provider, the data monitor connected with the care provider management system;
evaluating the received data from the remote monitoring system using the data monitor; and
conveying an evaluation from the data monitor to the care provider management system.

29. The method of claim 28, wherein evaluating the received data comprises accessing information in a data storage area, the information including patient medical records.

30. The method of claim 29, further comprising comparing the data received from the remote monitoring system with information in the data storage area in order to determine whether the received data meets pre-set expectations.

31. The method of claim 30, further comprising accessing a scheduling storage area for storing patient scheduling information.

32. The method of claim 31, further comprising adjusting a patient appointment schedule using a scheduling module.

33. The method of claim 31, wherein adjusting a patient appointment schedule comprises canceling a patient appointment.

34. The method of claim 31, further comprising adjusting a patient medication schedule using a scheduling module.

35. The method of claim 31, further comprising providing scheduling notification to a patient upon triggering a scheduling action.
